(19) United States
(12) Patent Application Publication
(10) Pub. No.: US 2016/0228288 A1
(43) Pub. Date: Aug. 11, 2016

(54) THERMAL COMPRESSION THERAPY DEVICE

(71) Applicant: TRIAD SPORTS, INC. dba BOWNET, CAMARILLO, CA (US)

(72) Inventors: DAVID NELSON, CAMARILLO, CA (US); JOHN LUCAS, CAMARILLO, CA (US)

(21) Appl. No.: 14/617,549

(22) Filed: Feb. 9, 2015

(51) Int. Cl. A61F 7/10; A61H 7/00

(52) U.S. Cl. CPC A61F 7/10; A61H 7/001; A61F 2007/0019

(57) ABSTRACT

A thermal compression therapy device comprising a garment and at least one thermal pack. The garment sized for placement around the torso and over the shoulders of an individual for applying a thermal treatment, the garment comprising a first region comprising a first cup area configured to cover a first chest area of the individual, a second region comprising a second cup area configured to cover a second chest area of the individual, at least one first shoulder strap configured to extend from said first region to said second region, at least one second shoulder strap configured to extend from said second region to said first region, wherein said first and second regions adapted to be removably coupled to each other, and at least one first opening in said first cup area configured to receive a first thermal pack.

### BACKGROUND OF THE INVENTION

1. Field of the Invention

This invention relates to a compression therapy device adapted to provide thermal compression therapy. More specifically, the invention is directed to a garment that is configured to provide thermal compression therapy to at least a portion of a chest area of an individual.

2. Description of the Related Art

Cryotherapy, which is the application of cold to an injured area, is a treatment used to manage the magnitude of the inflammatory process, blood flow, initial swelling, secondary injury and pain. The use of ice packs is widespread because of their effectiveness, convenience, low cost, and ease of transportation.

Ice pack therapy is a treatment of cold temperatures to an injured area of the body. An ice pack is placed over an injured area and is intended to absorb heat of a closed traumatic or edematous injury by using conduction to transfer thermal energy. The physiologic effects of cold application include immediate vasoconstriction with reflexive vasodilation, decreased local metabolism and enzymatic activity, and decreased oxygen demand. Cold decreases muscle spindle fiber activity and slows nerve conduction velocity, therefore it is often used to decrease spasticity and muscle guarding. It is commonly used to alleviate the pain of minor injuries, as well as decrease muscle soreness. The use of ice packs in treatment decreases the blood flow most rapidly at the beginning of the cooling period. Ice is a very popular modality for treatment in injuries and muscle repair following any activity.

Cold compression therapy is typically used to reduce pain and swelling from a sports or activity injury to soft tissues and recommended by surgeons following surgery. The therapy is especially useful for sprains, strains, pulled muscles and pulled ligaments. Cold Compression is a combination of cryotherapy and static compression, commonly used for the treatment of pain and inflammation after acute injury or surgical procedures. The use of cryotherapy in acute injury management lowers the temperature of the injured tissue, which reduces the tissue's metabolic rate and helps the tissue to survive the period following the injury. Static compression is often used in conjunction with cryotherapy for the care of acute injuries, which increases external pressure on the tissue to prevent swelling. This occurs by hindering fluid loss from the vessels in the injured area, making it more difficult for fluids to accumulate. Ice with compression is significantly colder than ice alone due to improved skin contact and increased tissue density caused by extended static compression. Tissue reaches its lowest temperature faster and the tissue maintains its cool even after treatment ends.

Cold compression wraps can be used to apply the ice or cold to the desired body part. Many of the wraps available use adjustable elastic straps to aid in compression over the injured areas. Some wraps are used to hold an ice pack at the desired area and require the wrap to be wrapped around the desired area, i.e. torso, arm, or leg, whereas other wraps have a freezable ice pack that can be reused integrated into the wrap or have a pocket or compartment to receive the ice pack. These wraps require that the wrap be wrapped around the desired area in order to hold the ice pack in place. Wrapping an ice pack around a particular body part can require an excessive amount of wrapping in order to hold the ice pack at the desired area due to gravity and/or the desired area being at a point of the body that does not easily allow it to be wrapped, such as a shoulder or the torso. The wrapping could also be required to be wrapped around multiple body parts, several times, in order to properly secure the ice pack to the desired area, which could lead to discomfort. However, wrapping the wrap around the desired area could lead to inconsistent pressure being applied to the desired area due to size and/or shape of the wrap.

Plastic film wrap can also be used to wrap the ice pack around a desired area and is easy to apply and low in cost. However, the plastic film wrap must be discarded afterwards and is not reusable. Reusable wraps, such as bandages, are available in the marketplace, but still require that the bandage be wrapped around the body part to hold the ice pack in place.

Some wraps are molded in the shape of particular body parts, such as knee/elbow joints, shoulder, neck, arms, etc., and are configured to hold the ice pack at the desired area. These molded wraps come in various sizes such as small, medium, large, extra-large, but are not arranged to fit various body sizes.

Securing an ice pack to certain body parts can be difficult due to the location of the particular body part. For example, applying an ice pack to the chest areas of individuals can be accomplished by the individuals holding the ice pack themselves, wrapping a bandage or wrap multiple times around the torso and/or shoulders, or wearing a compression garment, such as a compression bra that has compartments to receive ice packs. Individuals holding the ice pack themselves can be bothersome because the individual must physically hold and apply the ice pack onto the desired area. Individuals could get tired and/or not properly apply the ice pack to the desired area. Wrapping a bandage or wrap multiple times could hold the ice pack to the chest area, but could require the wrap being wrapped around multiple body parts which could cause discomfort. Also, individuals would likely need the assistance of another person to wrap the ice pack to the chest area using a bandage or wrap. A compression garment, such as the compression bra, allows individuals to apply the ice pack to the chest area without having to hold the ice pack themselves and/or without the assistance of another person, thereby freeing the hands and allowing the individual to go about their lives. However, compression bras come in a variety of sizes ranging from small to extra-large, and are not configured to fit various body sizes. The compression bras might be slightly adjustable, but would not allow the sizing to vary between a small and an extra-large.

Thus, what is needed is a compression therapy device that is reusable and is arranged to fit various body sizes.

### SUMMARY

A thermal compression therapy device for chest areas of an individual is presented which overcomes the problems noted above. In one embodiment, the thermal compression therapy device comprising a garment sized for placement around the torso and over the shoulders of an individual for applying a thermal treatment, the garment comprising a first region comprising a first cup area configured to cover a first chest area of the individual, a second region comprising a second cup area configured to cover a second chest area of the individual, at least one first shoulder strap configured to extend from said first region over a first shoulder of said individual to said second region, at least one second shoulder strap configured to extend from said second region over a second shoulder of said individual to said first region, wherein said first region and said second region adapted to be removably coupled to each other, and at least one first opening in said first cup area configured to receive a first thermal pack, said first thermal pack configured to be interposed between at least part of said first chest area and said first cup area.

In another embodiment, the thermal compression therapy device comprising a garment sized for placement around the torso and over the shoulders of an individual for applying a thermal treatment, the garment comprising a first region comprising a first cup area, a first apex extending from an upper portion of said first cup area, a first shoulder strap extending from a lower portion of said first cup area, a first back band extending from a lower portion of the first cup area proximate said first shoulder strap, and a first front panel extending from a lower portion of said first cup area opposite said first back band and said first shoulder strap. The garment further comprising a second region comprising a second cup area, a second apex extending from an upper portion of said second cup area, a second shoulder strap extending from a lower portion of said second cup area, a second back band extending from a lower portion of said second cup area proximate said second shoulder strap, a second front panel extending from a lower portion of said second cup area opposite said second back band and said second shoulder strap, wherein said first region and said second region are adapted to be removably coupled to each other, wherein said first cup area is configured to receive a first thermal pack, said first cup area adapted to hold said first thermal pack within said first cup area, and wherein said second cup area is configured to receive a second thermal pack, said second cup area adapted to hold said second thermal pack within said second cup area.

In yet another embodiment, the thermal compression therapy device for chest areas of an individual comprising a garment sized for placement around the torso and over the shoulders of said individual, the garment comprising a first region comprising a first cup area, a second region comprising a second cup area, and a first shoulder strap configured to extend from said first region over a first shoulder of said individual to said second region, and a second shoulder strap configured to extend from said second region over a second shoulder of said individual to said first region, wherein said first and second shoulder straps overlap in a crisscross arrangement, wherein said first region and said second region are adapted to be removably coupled to each other. The thermal compression therapy device further comprising a first thermal pack received by said first cup area and configured to be interposed between at least part of a first chest area of said individual and said first cup area, and a second thermal pack received by said first cup area and configured to be interposed between at least part of a second chest area of said individual and said second cup area.

These and other features, aspects and advantages of the invention will become better understood with reference to the following description and claims.

### BRIEF DESCRIPTION OF THE DRAWINGS

FIG. 1 is a perspective view of a thermal compression therapy device according to an embodiment of the invention applied to a torso of an individual.

FIG. 2 is a front view of the thermal compression therapy device of FIG. 1.

FIG. 3 is a rear view of the thermal compression therapy device of FIG. 1.

FIG. 4 is a partial cross sectional view of the thermal compression therapy device of FIG. 1.

FIG. 5 is a front view of a garment according to an embodiment of the invention.

FIG. 6 is a rear view of the garment of FIG. 5.

FIG. 7a is a front view of a first region of the garment of FIG. 5.

FIG. 7b is a rear view of the first region of the garment of FIG. 5.

FIG. 8a is a front view of a second region of the garment of FIG. 5.

FIG. 8b is a rear view of the second region of the garment of FIG. 5.

### DETAILED DESCRIPTION

Embodiments of the invention provide a thermal compression therapy device that is configured to provide thermal compression therapy to at least a portion of a chest area of an individual. The thermal compression therapy device according to the invention can be arranged in many different ways and with many different components, and is generally arranged to provide a garment sized for placement around the torso and over the shoulders of an individual for applying a thermal treatment. The garment adapted to alter its size to fit various body sizes.

The invention is described herein with reference to certain embodiments, but it is understood that the invention can be embodied in many different forms and should not be construed as limited to the embodiments set forth herein. In particular, the invention is described below in regards to certain thermal compression therapy devices in different configurations, but it is understood that the invention can be used to apply thermal compression therapy to many other areas of an individual and can have many different configurations. The components can have different shapes and sizes beyond those shown in the figures or discussed herein.

It is to be understood that when an element or component is referred to as being “on” another element or component, it can be directly on the other element or intervening elements may also be present. Furthermore, relative terms such as “between”, “within”, “below”, and similar terms, may be used herein to describe a relationship of one element or component to another. It is understood that these terms are intended to encompass different orientations of the device in addition to the orientation depicted in the figures.

Although the terms first, second, etc. may be used herein to describe various elements or components, these elements or components should not be limited by these terms. These terms are only used to distinguish one element or component from another. Thus, a first element discussed herein could be termed a second element without departing from the teachings of the present application. It is understood that actual devices embodying the invention can be arranged in many different ways with many more features and elements beyond what is shown in the figures.

Embodiments of the invention are described herein with reference to illustrations that are schematic illustrations. As such, the actual thickness of elements can be different, and variations from the shapes of the illustrations as a result, for example, of manufacturing techniques and/or tolerances are expected. Thus, the elements illustrated in the figures are schematic in nature and their shapes are not intended to illustrate the precise shape of a region of a device and are not intended to limit the scope of the invention.

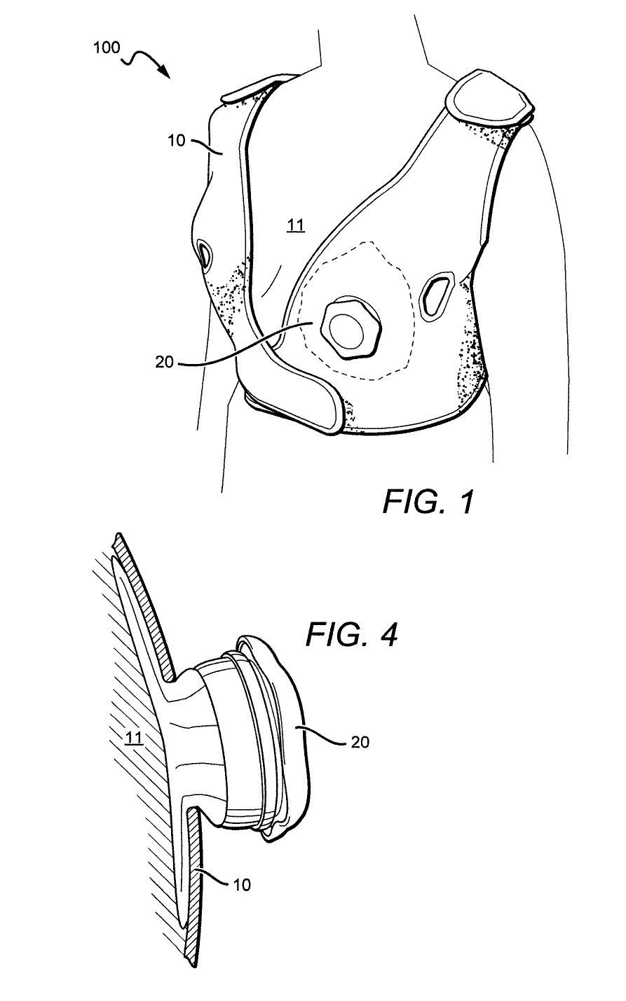

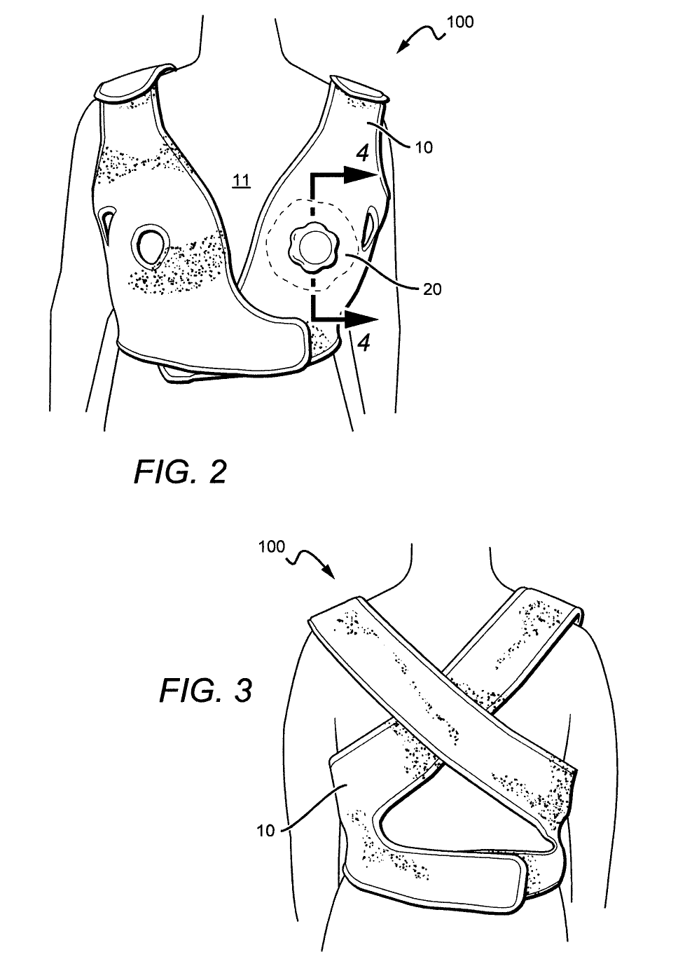

FIGS. 1-4 show one embodiment of a thermal compression therapy device 100 according to the invention. The thermal compression therapy device 100 comprises a garment 10 and at least one thermal pack 20. The garment 10 is sized for placement around the torso and over the shoulders of an individual and is arranged to apply thermal therapy to the chest area of the individual. The garment 10 is configured to receive at least one thermal pack 20 and hold the at least one thermal pack 20 in place, wherein the at least one thermal pack 20 is interposed between the garment 10 and at least part of a chest area 11 of an individual. In one embodiment, the at least one thermal pack 20 can receive a cold substance, such as but not limited to ice or any other cold or frozen substance, in order to provide cold therapy to the chest area 11 of the individual. In another embodiment, the at least one thermal pack 20 can receive a heated substance, such as but not limited to hot water or any other heated substance, in order to provide heat therapy to the chest area 11 of the individual.

With reference to FIG. 4, when the garment 10 is worn by the individual, the at least one thermal pack 20 is removably coupled to the garment 10 and is interposed between at least part of the chest area 11 and an inner surface of the garment 10. The at least one thermal pack 20 is able to substantially conform to the shape of the chest area 11 of the individual. In one embodiment, the at least one thermal pack 20 can substantially conform to a lower portion of the chest area 11 of the individual, whereas in other embodiments, the at least one thermal pack 20 can substantially conform to an upper portion of the chest area 11. In yet other embodiments, the at least one thermal pack 20 can substantially conform to a substantial portion of the upper and lower portion of the chest area 11. The at least one thermal pack 20 can be a conventional reusable ice pack bag known in the art. However, the at least one thermal pack 20 is not intended to be limited to the conventional reusable ice pack bags. The garment 10 is adapted to be adjustable such that the sizing of the garment 10 could either increase and/or decrease to fit the torso of the individual. At least one advantage of the invention is that the size of the garment 10 can be adjusted to fit various different body shapes. This allows the garment 10 to be adjusted to provide a custom fit to each individual. Furthermore, the adjustability of the garment 10 allows for control of the compression force applied on the torso and/or the compression of the at least one thermal pack 20 applied onto the chest area 11 of the individual. Thereby allowing users to have full control of how the garment 10 fits their body.

The garment 10 is adapted to be worn around the torso and over the shoulders of the individual in order to provide thermal therapy to the chest area 11 of the individual. In one embodiment, the chest area 11 is a breast area of the individual and the garment 10 is configured to provide thermal therapy to the breast area of the individual. When individuals have surgical procedures conducted in the chest area, they can experience pain and swelling in the chest area after the surgical procedure. Application of an ice pack to the chest area is typically used to reduce swelling and/or reduce pain. However, conventional ice packs have to be held in place on the chest area by the individual using their hand or are wrapped to the chest area using plastic film and/or a bandage. In both of these instances, the ice pack cannot be securely held against the chest area or it could fall out when the individual stands up and/or moves. Also, wrapping the bandage or film around the torso to hold the ice pack in place could lead to discomfort. The garment 10 is configured to securely position and hold the at least one thermal pack 20 to the chest area to provide thermal compression therapy. The garment also provides the individual with freedom of movement and use of the arms because the garment 10 holds the at least one thermal pack 20 to their chest area. The individual does not have to physically hold the at least one thermal pack 20 to their chest area, and can stand and/or walk around without concern of the thermal pack 20 falling or shifting position.

The garment 10 is sized for placement around the torso and over the shoulders of the individual. The garment 10 is arranged to allow the individual to have freedom of movement while wearing the garment and applying thermal treatment to the chest area of the individual. The garment 10 is configured so that the weight of the garment 10 and the at least one thermal pack 20 can be distributed to both shoulders of the individual. This prevents the situation where only one shoulder of the individual would bear the load of the garment 10 and the at least one thermal pack 20 while the device 100 is being used. At least one advantage is that distributing the load to both shoulders of the individual reduces strain on the back of the individual, which thereby reduces the likelihood of the individual sustaining an injury from using the device 100.

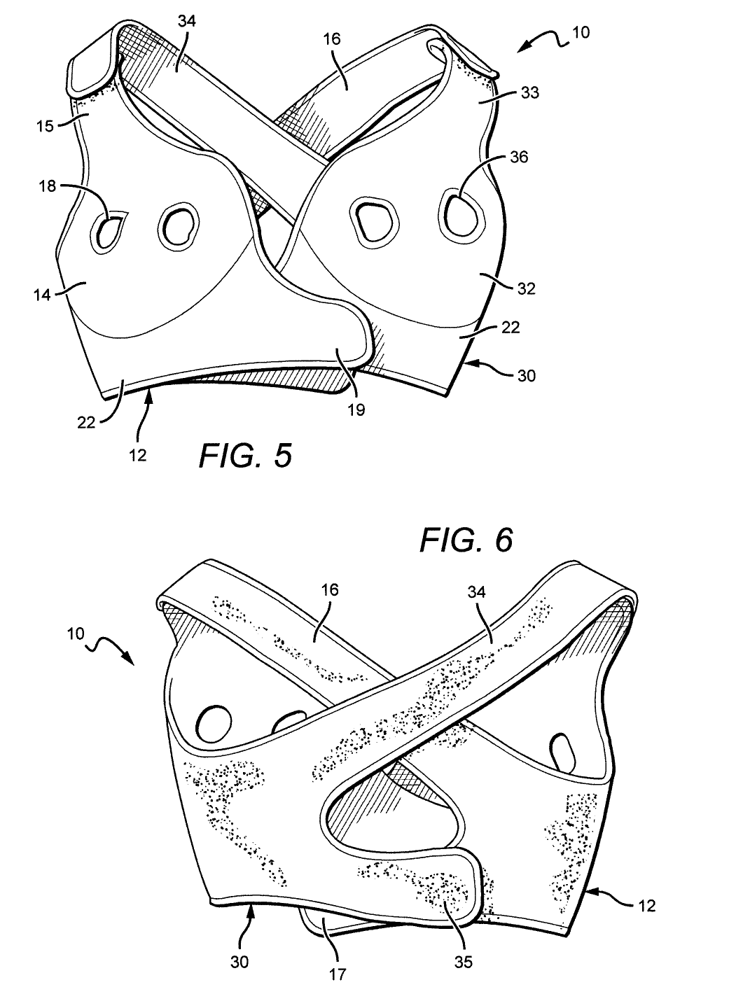

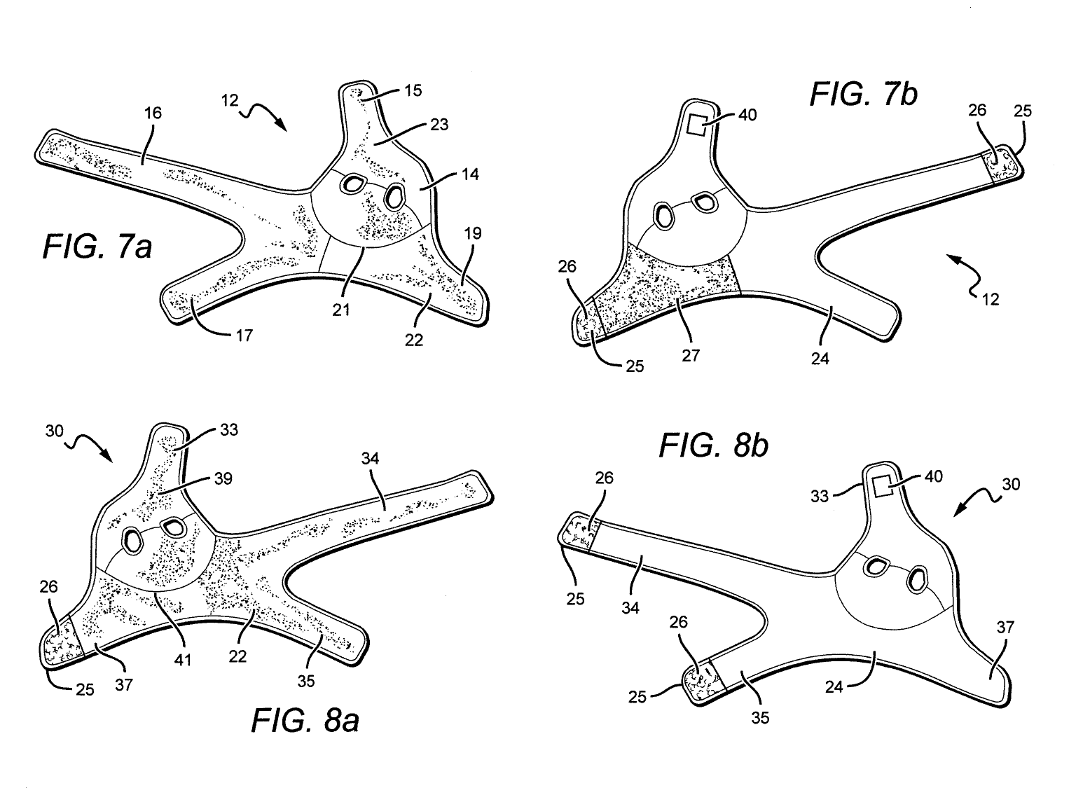

FIGS. 5-8b show one embodiment of the garment 10 according to the invention. For the same or similar features, the same reference numbers will be used throughout the application herein. The garment 10 comprises a first region 12 and a second region 30. The first region 12 comprises a first cup area 14 that is configured to cover a first chest area 3 of the individual. The second region 30 comprises a second cup area 32 that is configured to cover a second chest area 5 of the individual. In one embodiment, the first cup area 14 and the second cup area 32 are shaped to contour a respective first and second breast areas of the individual. The first and second cup areas 14, 32 can be comprised of one or more stitched panels. The one or more stitched panels are assembled such that the first and second cup areas 14, 32 are shaped to contour the respective first and second chest area and/or breast area of the individual. In other embodiments, the first and second cup areas 14, 32 can comprise a general cup-like shape.

The garment 10 further comprises at least one first shoulder strap 16 configured to extend from the first region 12 over a first shoulder of the individual to the second region 30. At least one second shoulder strap 34 is configured to extend from the second region 30 over a second shoulder of the individual to the first region 12. The at least one first shoulder strap 16 and the at least one second shoulder strap 34 extend to the opposing shoulder of the individual such that the at least one first and second shoulder straps 16, 34 overlap in a crisscross arrangement. The crisscross arrangement allows the weight of the garment 10 and the at least one thermal pack 20 to be distributed to both shoulders of the user, instead of the shoulders bearing the load of its respective first or second region 12, 30. The crisscross arrangement also provides back support while the individual wears the device 100 such that the individual does not sustain back strain and/or injuries from wearing the device 100.

The first region 12 further comprises at least one first opening 18 in the first cup area 14. The first opening 18 is configured to receive at least one thermal pack 20. The at least one thermal pack 20 received by the first opening 18 is configured to be interposed between at least part of the first chest area 3 (not shown) and the first cup area 14. The first opening 18 is smaller in size than that of the at least one thermal pack 20, and is adapted to expand its size in order to receive the thermal pack 20. Once the thermal pack 20 is received by the at least one first opening 18, the size of the at least one first opening 18 substantially returns to the original size, and in doing so, imparts a compression force onto the thermal pack 20. The at least one first opening 18 does not completely return to its original size when it receives the thermal pack 20, because not all of the thermal pack 20 is completely within the first cup area 14. In some embodiments, at least part of the thermal pack 20 remains outside of the first cup area 14 and since the size of the thermal pack 20 is bigger than that of the at least one first opening 18, the at least one first opening 18 resultantly applies a compression force onto the thermal pack 20 that is within the at least one first opening 18. This compression force holds the thermal pack within the first cup area 14. The compression force the garment 10 applies to the torso of the individual also contributes in holding the thermal pack 20 within the first cup area 14, when the garment 10 is in use.

The first region 12 can be comprised of material exhibiting flexible, resilient, and elastic properties. The material used for the first region 12 allows the at least one first opening 18 to easily expand to receive the at least one thermal pack 20. The elastic properties of the first region 12 allows the at least one first opening 18 to repeatedly return to its original size after continued usage. However, when the at least one thermal pack 20 is within the at least one first opening 18, the at least one first opening 18 substantially returns to its original size because of the presence of the at least one thermal pack 20. The at least one first opening 18 applies a compression force around the at least one thermal pack 20 and holds the at least one thermal pack 20 within the first cup area 14. In one embodiment, the material used for the first region 12 can be neoprene. However, other similar materials can be used and the invention is not intended to be limited to neoprene. In other embodiments, the at least one thermal pack 20 can be completely inserted into the first cup area 14 and can be held in place by a compression force applied by the garment 10 onto the torso of the individual. In yet other embodiments, the at least one thermal pack 20 is not present and the garment 10 is worn alone and used as a compression garment to apply a compression force on the chest area and/or the torso of the individual. Compression therapy without a thermal treatment is often used after surgery to assist during the healing process, and can reduce swelling. The individual can use the garment 10 in a combination of compression therapy and thermal compression therapy without having to use multiple garments and/or bandages specially designed for each type of therapy.

The second region 30 further comprises at least one second opening 36 in the second cup area 32. The at least one second opening 36 is configured similarly as the at least one first opening 18 of the first cup area 14, discussed above, and can receive at least one thermal pack 20 in a similar manner as the at least one first opening 18 of the first cup area 14. At least one thermal pack 20 is received by the at least one second opening 36 and is configured to be interposed between at least part of the second chest area 5 (not shown) and the second cup area 32. The at least one second opening 36 is also smaller than the at least one thermal pack 20. Once the thermal pack 20 is received by the at least one second opening 36, the size of the at least one second opening 36 substantially returns to its original shape and imparts a compression force onto the thermal pack 20, similarly as in the at least one first opening 18. This compression force holds the thermal pack 20 within the second cup area 32. The compression force the garment 10 applies to the torso of the individual also contributes in holding the thermal pack 20 within the second cup area 32, when the garment 10 is in use. The second region 30 can also be comprised of the same material as the first region 12.

The at least one first shoulder strap 16 is configured to extend from the first region 12 towards the second region 30. In one embodiment the at least one first shoulder strap 16 extends from a lower portion 21 of the first cup area 14. The at least one first shoulder strap 16 comprises an end 25 that is adapted to be removably coupled to the second region 30. In one embodiment, the end 25 comprises hook fasteners that fasten the at least one first shoulder strap 16 to the second region 30. The second region 30 comprises an inner surface 24 and an outer surface 22, wherein the outer surface 22 is adapted to receive the end 25 of the first shoulder strap 16 in order to removably couple the first shoulder strap 16 to the second region 30. The outer surface 22 of the second region 30 comprises a plurality of loops such that the hook fasteners of the end 25 can securely fasten the first shoulder strap 16 to the second region 30. A substantial portion of the outer surface 22 of the second region 30 comprises a plurality of loops. In one embodiment, the outer surface 22 of the second region 30 is formed of a looped material, and the inner surface 24 of the second region 30 is comprised of material exhibiting flexible, resilient, and elastic properties, such as but not limited to neoprene. While in other embodiments, only part of outer surface 22 comprises the looped material. The outer surface 22 of the second region allows the end 25 of the first shoulder strap 16 to be removably coupled to the second region 30 at substantially any point of the outer surface 22. This allows the positioning of the first shoulder strap 16 to be adjusted in order to customize the size and/or the fit of the garment 10. The end 25 of the first shoulder strap 16 can comprise many different fastening devices, such as but not limited to buttons, clips, or the like, and is not intended to be limited to hook fasteners. The outer surface 22 can comprise many different complementary fastening devices, such as but not limited to button holes, clasps, or the like, and is not intended to be limited to looped material or comprising a plurality of loops.

The at least one second shoulder strap 34 is configured to extend from the second region 30 towards the first region 12. In one embodiment the at least one second shoulder strap 34 extends from a lower portion 41 of the second cup area 32. The at least one second shoulder strap 34, is configured in a manner similar to the first shoulder strap 16, and also comprises an end 25 adapted to be removably coupled to the first region 12. In one embodiment, the end 25 of the at least one second shoulder strap 34 can also comprise hook fasteners that fasten the at least one second shoulder strap 34 to the first region 12. The first region 12 comprises an inner surface 24 and an outer surface 22, similar to the second region 30 discussed above. The outer surface 22 of the first region 12 is adapted to receive the end 25 of the second shoulder strap 34 in order to removably couple the second shoulder strap 34 to the first region 12. The outer surface 22 of the first region 12 allows the end 25 of the second shoulder strap 34 to be removably coupled to the first region 12 at substantially any point of the outer surface 22 of the first region 12. This also allows the positioning of the second shoulder strap 34 on the outer surface 22 of the first region 12 to be adjustable in order to customize the size and/or fit of the garment 10. At least one advantage of the invention is that the adjustability of the first and second shoulder straps 16, 34 allows the garment 10 to be size adjustable for various different body types. The adjustability of the first and second shoulder straps 16, 34 also allows the compression applied by the garment 10 onto the chest areas of the individual to be altered.

The first region 12 can further comprise a first apex 15, a first back band 17, and a first front panel 19. The second region 30 can further comprise a second apex 33, a second back band 35, and a second front panel 37. The first and second apexes 15, 33, first and second back bands 17, 35, and first and second front panels 19, 37 are configured to removably couple the first and second regions 12, 30 to each other.

The first apex 15 extends from an upper portion 23 of the first cup area 14. The first apex 15 is adapted to receive the second shoulder strap 34 such that the second shoulder strap 34 is removably coupled to the first region 12. The first apex provides a surface to receive the end 25 of the second shoulder strap 34 in order to removably couple the second shoulder strap 34 to the first region 12. The end 25 of the second shoulder strap 34 is fastened to the outer surface 22 of the first apex 15. As discussed above, the outer surface 22 of the first region 12 can comprise a plurality of loops 27. As such, the outer surface 22 of the first apex 15 is adapted to receive the hook fasteners 26 of the end 25 of the second shoulder strap 34. The second apex 33 is configured similarly to the first apex 15. The second apex 33 extends from an upper portion 39 of the second cup area 32. The second apex 33 is adapted to receive the first shoulder strap 16, such that the first shoulder strap 16 is removably coupled to the second region 30. The second apex 33 provides a surface to receive the end 25 of the first shoulder strap 16 in order to removably couple the first shoulder strap 16 to the second region 30. The end 25 of the first shoulder strap 16 is fastened to the outer surface 22 of the second apex 33. The outer surface 22 of the second apex 33 comprises a plurality of loops 27 to receive the hook fasteners of the end 25 of the first shoulder strap 16.

The first apex 15 and the second apex 33 can further comprise an orientation marking 40 which identifies which side the first and second regions 12, 30 are to be positioned when the garment 10 is constructed. The orientation markings 40 also indicate which side of the torso of the individual the first and second regions 12, 30 are to be located when the garment 10 is worn. In one embodiment, the orientation markings 40 can be on the inner surface 24 of the first and second apexes 15, 33. In other embodiments, the orientation markings 40 can be on the outer surface 22 of the first and second regions 12, 30. The orientation markings 40 can be comprised of one or more digits, letters, characters, figures or the like. In one embodiment, the orientation marking 40 on the inner surface 24 of the first apex 15 is the letter “R” indicating that the first region 12 is configured to be worn on the right side of the torso, while the orientation marking 40 on the inner surface 24 of the second apex 33 is the letter “L” indicating that the second region 30 is configured to be worn on the left side of the torso.

The hook and loop fastener combination securely fastens the second shoulder strap 34 to the first apex 15 and the first shoulder strap 16 to the second apex 33. The first and second shoulder straps 16, 34 can be removably coupled to the respective apex 33, 15 at substantially any point of the outer surface 22 of the respective apex 33, 15. The adjustability of coupling the first and second shoulder straps 16, 34 to the respective apex 33, 15 allows the compression applied on the torso by the garment 10 to be adjusted. The adjustability of the first and second shoulder straps 16, 34 can also adjust the support each first and second cup area 14, 32 provide when the garment 10 is worn. The adjustability of the first and second shoulder straps 16, 34 also provides further customization of the garment 10 such that the size of the garment 10 can be adjusted for various body sizes.

The first back band 17 extends from a lower portion 21 of the first cup area 14 and is proximate the at least one first shoulder strap 16. The second back band 35 extends from a lower portion 41 of the second cup area 32 and is proximate the at least one second shoulder strap 34. The first back band 17 is adapted to receive the second back band 35 of the second region 30, wherein the second back band 35 is removably coupled to the first region 12. The first back band 17 provides a surface to receive an end 25 of the second back band 35 in order to removably couple the second back band 35 to the first region 12. The end 25 of the second back band 35 is adapted to be fastened to the outer surface 22 of the first back band 17. The end 25 of the second back band 35 can be removably coupled at substantially any point along the length of the outer surface 22 of the first back band 17. The first and second back bands 17, are configured to form the lower support portion of the garment 10 when the first and second regions 12, 30 are coupled. When the first and second back bands 17, 35 are fastened, they assist in securing the garment 10 to the torso of the individual. The first and second back bands 17, 35 can also assist in positioning the first and second cup areas 14, 32 to cover the respective first and second chest areas 3, 5 of the individual. Adjusting the position where the end 25 of the second back band 35 is removably coupled to the first back band 17 can also adjust the compression force the garment 10 applies to the torso of the individual. Furthermore, the first and second back bands 17, 35 are adapted to adjust the size of the garment 10 for various body sizes.

The first front panel 19 extends from the lower portion 21 of the first cup area 14 opposite the first back band 17 and the first shoulder strap 16. The second front panel 37 extends from the lower portion 41 of the second cup area 32 opposite the second back band 35 and the second shoulder strap 34. The first front panel 19 is adapted to be removably coupled to the second front panel 37 of the second region 30. The first front panel 19 comprises an end 25 that is configured to be received by the second front panel 37. The second front panel 37 provides a surface to receive the end 25 of the first front panel 19. The end 25 of the first front panel 19 is adapted to be fastened to the outer surface 22 of the second front panel 37. The end 25 of the first front panel 19 can comprise hook fasteners 26 and the outer surface 22 of the second front panel 37 can comprise a plurality of loops 27, such that the first front panel 19 can be removably coupled to the second front panel 37. In one embodiment, the end 25 of the first front panel 19 is on the inner surface 24 of the first front panel 19. However, in other embodiments, the end 25 of the first front panel 19 can be on the outer surface 22 of the first front panel 19. The end 25 of the first front panel 19 can be coupled to substantially any part of the outer surface 22 of the second front panel 37. The first front panel 19 is configured to receive the second front panel 37. The second front panel 37 comprises an end 25 and is configured to be received by the first front panel 19. The first front panel 19 provides a surface to receive the end 25 of the second front panel 37. The end 25 of the second front panel 37 is adapted to be fastened to the inner surface 24 of the first region 12. In one embodiment the end 25 of the second front panel 37 is on the outer surface 22 of the second front panel 37. However, in other embodiments, the end 25 of the second front panel 37 can be on the inner surface 24 of the second front panel 37. Part of the inner surface 24 of the first region 12 comprises a plurality of loops 27 on the inner surface 24 of the first front panel 19. The end 25 of the second front panel 37 can comprise hook fasteners 26 such that the end 25 of the second front panel 37 can be removably coupled to the inner surface 24 of the first front panel 19. The end 25 of the second front panel 37 can be coupled to substantially any part of the inner surface 24 that comprises the plurality of loops 27 of the first front panel 19. This configuration results in the first front panel 19 overlapping the second front panel 37.

At least one advantage of the invention is that the overlapping configuration of the front panels provides for only one manner of properly constructing the garment 10, and eliminates confusion as to proper construction of the garment 10. For example, proper construction of the garment 10 requires the first region 12 and the second region 30 to be removably coupled to each other, wherein the second front panel 37 is removably coupled to the inner surface 24 of the first front panel 19, and the first front panel 19 is removably coupled to the outer surface 22 of the second front panel 37. The ends 25 of the first and second front panels 19, 37 are configured such that the first and second front panels 19, 37 can be removably coupled to each other with ease. Furthermore, proper construction of the garment 10 further requires the second back band 35 to be removably coupled to the outer surface 22 of the first back band 17. The end 25 of the second back band 35 is on the inner surface 24 of the second back band 35 and allows the second back band 35 to be easily removably coupled to the first back band 17. Another advantage of the invention is that the overlapped first and second front panels provides structural support to the garment 10 and can also assist in securing the garment 10 to the torso of the individual. The first front panel 19 being removably coupled to the second front panel 37, and the second front panel 37 being removably coupled to the first front panel 19 securely fastens the front panels 19, 37 together and allows the garment 10 to support the load of one or more thermal packs 20 that may be received by one or both of the first and second cup areas 14, 32. Additionally, the coupled first and second front panels 19, 37 also provide support to the first and second chest areas 3, 5 within the respective first and second cup areas 14, 32. Furthermore, the coupling of the first and second front panels 19, 37 can be adjusted in order to adjust the compression force the garment 10 applies to the torso. The first and second front panels 19, 37 are also adapted to adjust the size of the garment 10 for various body sizes.

Yet another advantage of the invention is that the first and second front panels 19, 37 are adapted to adjust the separation between the first cup area 14 and the second cup area 32. In some instances, the first and second front panels 19, 37 can be adjustable to position the first and second cup areas 14, to substantially correspond to the separation between the first and second chest areas 3, 5 of the individual. Once the desired positioning of the first and second cup areas 14, 32 are set by the individual, the first and second back bands 17, 35, and the first and second shoulder straps 16, 34 can be adjusted to set the desired compression the garment 10 applies to the torso of the individual.

The garment 10 is highly adjustable and is adapted to accommodate various body shapes and sizes. The adjustability of the garment 10 allows the garment 10 to essentially be a universal sized garment or a one size fits all garment. For example, the size of the garment 10 can be adjusted to range from a size small to extra-large. The size of the garment 10 can be varied by adjusting the coupling of any one or all of the following: the first and second front panels 19, 37, the first and second back band 17, 35, and/or the first and second shoulder straps 16, 34. The compression force applied to the torso by the garment 10 can also be increased/decreased by adjusting the coupling of any one or all of the following: the first and second front panels 19, 37, the first and second back band 17, 35, and/or the first and second shoulder straps 16, 34. The garment 10 can be worn alone without any thermal packs 20 and used as a compression garment. The garment 10 can be comprised of neoprene, as discussed above, which is a breathable material and allows the garment 10 to be in contact with the skin of the individual. The garment 10 can be worn underneath clothing, or over clothing. The garment 10 is easily washable and reusable. The first and second region 12, 30 being removably coupled allows the garment 10 to be disassembled prior to cleaning and/or storage. The first and second regions 12, 30 being removably coupled allows for any damaged region to be easily replaced without having to replace the entire garment 10. Also, since the garment 10 is made in one size there are no instances where users obtain a replacement region in the incorrect size. Furthermore, the adjustability of the garment also provides individuals with a cost-effective solution. Some compression garments are custom made to fit a specific individual, and these custom-fitted garments can be expensive. The device 100 allows individuals to obtain a compression garment that can be used to provide a thermal treatment and/or a compression treatment that is adjustable to custom fit the individuals without having to purchase a custom made compression garment, which can reduce expenses incurred by individuals

While particular embodiments of the invention have been shown and described, numerous variations and alternate embodiments will occur to those skilled in the art. Accordingly, it is intended that the invention be limited only in terms of the appended claims.

We claim:

1. A garment sized for placement around the torso and over the shoulders of an individual for applying a thermal treatment, said garment comprising:
a first region comprising a first cup area configured to cover a first chest area of said individual;
a second region comprising a second cup area configured to cover a second chest area of said individual; and
at least one first shoulder strap configured to extend from said first region over a first shoulder of said individual to said second region, and at least one second shoulder strap configured to extend from said second region over a second shoulder of said individual to said first region, said first region and said second region adapted to be removably coupled to each other;
at least one first opening in said first cup area configured to receive a first thermal pack, said first thermal pack configured to be interposed between at least part of said first chest area and said first cup area.

2. The garment of claim 1, further comprising at least one opening in said second cup area configured to receive a second thermal pack, said second thermal pack configured to be interposed between at least part of said second chest area and said second cup area.

3. The garment of claim 2, wherein the positioning of said at least one second shoulder strap on said first region is adapted to be altered to adjust a compression force applied by said garment onto the torso of said individual.

4. The garment of claim 2, wherein said at least one opening in said second cup area is adapted to apply a compression force onto said second thermal pack in order to maintain said second thermal pack within said second cup area.

5. The garment of claim 1, wherein the positioning of said at least one first shoulder strap on said second region is adapted to be altered to adjust a compression force applied by said garment onto the torso of said individual.

6. The garment of claim 1, wherein said at least one opening in said first cup area is adapted to apply a compression force onto said first thermal pack in order to maintain said first thermal pack within said first cup area.

7. The garment of claim 1, wherein said at least one first shoulder strap is configured to be removably coupled to said second region and said at least one second shoulder strap is configured to be removably coupled to said first region, such that the positioning of said at least one first shoulder strap on said second region and said at least one second shoulder strap on said first region allows said garment to be size adjustable for a particular individual.

8. The garment of claim 1, wherein each of said first region and said second region comprises an outer surface and an inner surface, wherein said outer surface of each of said first region and said second region comprises a plurality of loops.

9. The garment of claim 8, wherein each of said at least one first and second shoulder straps comprises an end, each of said ends comprising hook fasteners, wherein said end of said at least one first shoulder strap is adapted to be removably coupled to said outer surface of said second region and said end of said at least one second shoulder strap is adapted to be removably coupled to said outer surface of said first region.

10. A garment sized for placement around the torso and over the shoulders of an individual for applying a thermal treatment, said garment comprising:
a first region comprising:
a first cup area;
a first apex extending from an upper portion of said first cup area;
a first shoulder strap extending from a lower portion of said first cup area;
a first back band extending from a lower portion of said first cup area proximate said first shoulder strap; and
a first front panel extending from a lower portion of said first cup area opposite said first back band and said first shoulder strap;
a second region comprising:
a second cup area;
a second apex extending from an upper portion of said second cup area;
a second shoulder strap extending from a lower portion of said second cup area;
a second back band extending from a lower portion of said second cup area proximate said second shoulder strap; and
a second front panel extending from a lower portion of said second cup area opposite said second back band and said second shoulder strap;
wherein said first region and said second region are adapted to be removably coupled to each other;
wherein said first cup area is configured to receive a first thermal pack, said first cup area adapted to hold said first thermal pack within said first cup area; and
wherein said second cup area is configured to receive a second thermal pack, said second cup area adapted to hold said second thermal pack within said second cup area.

11. The garment of claim 10, wherein said first shoulder strap, said first back band, and said first front panel extend toward said second region and are adapted to removably couple said first region to said second region.

12. The garment of claim 10, wherein said first shoulder strap is adapted to be removably coupled to said second apex, said first back band is adapted to be removably coupled to said second back band, and said first front panel is adapted to be removably coupled to said second front panel.

13. The garment of claim 12, wherein each of said first shoulder strap and said first front panel comprises an end, each of said ends comprising hook fasteners.

14. The garment of claim 13, wherein said second region comprises an outer surface, said outer surface comprising a plurality of loops such that said ends of said first shoulder strap and said first front panel are adapted to be removably coupled to said outer surface of said second region.

15. The garment of claim 10, wherein said second shoulder strap, said second back band, and said second front panel extend toward said first region and are adapted to removably couple said second region to said first region.

16. The garment of claim 10, wherein said second shoulder strap is adapted to be removably coupled to said first apex, said second back band is adapted to be removably coupled to said first back band, and said second front panel is adapted to be removably coupled to said first front panel.

17. The garment of claim 16, wherein each of said second shoulder strap, second back band, and second front panel comprises an end, each of said ends comprising hook fasteners.

18. The garment of claim 17, wherein said first region comprises an outer surface, said outer surface comprising a plurality of loops, wherein said ends of said second shoulder strap and said second back band are adapted to be removably coupled to said outer surface of said first region.

19. The garment of claim 17, wherein said first region comprises an inner surface, wherein part of said inner surface of said first region comprises a plurality of loops configured to receive said end of said second front panel, wherein said second front panel is adapted to be removably coupled to said first front panel.

20. The garment of claim 10, wherein said first cup area comprises at least one opening and said second cup area comprises at least one opening.

21. The garment of claim 20, wherein said at least one opening of said first cup area receives said first thermal pack and is adapted to maintain said first thermal pack within said first cup area.

22. The garment of claim 20, wherein said at least one opening of said second cup area receives said second thermal pack and is adapted to maintain said second thermal pack within said second cup area.

23. The garment of claim 10, wherein said first shoulder strap is removably coupled to said second apex, and said second shoulder strap is removably coupled to said first apex, such that said first and second shoulder straps overlap in a crisscross configuration.

24. The garment of claim 10, wherein said first back band is removably coupled to said second back band in order to provide structural support to said garment.

25. The garment of claim 24, wherein the coupling of said first and second back bands can be adjusted to allow said garment to be size adjustable for a particular individual.

26. The garment of claim 10, wherein said first and second front panels are removably coupled to each other such that said first front panel overlaps said second front panel, said first and second front panels adapted to provide structural support to said garment.

27. The garment of claim 26, wherein the coupling of said first and second front panels can be adjusted to allow said garment to be size adjustable for a particular individual.

28. A thermal compression therapy device for chest areas of an individual, comprising:
a garment sized for placement around the torso and over the shoulders of said individual, said garment comprising:
a first region comprising a first cup area;
a second region comprising a second cup area; and
a first shoulder strap configured to extend from said first region over a first shoulder of said individual to said second region, and a second shoulder strap configured to extend from said second region over a second shoulder of said individual to said first region, wherein said first and second shoulder straps overlap in a crisscross arrangement, wherein said first region and said second region are adapted to be removably coupled to each other;
a first thermal pack received by said first cup area and configured to be interposed between at least part of a first chest area of said individual and said first cup area; and
a second thermal pack received by said first cup area and configured to be interposed between at least part of a second chest area of said individual and said second cup area.

29. The thermal compression therapy device of claim 28, wherein said first cup area comprises at least one opening and said second cup area comprises at least one opening, wherein said first thermal pack is received by said at least one opening of said first cup area and said second thermal pack is received by said at least one opening of said second cup area.

30. The thermal compression therapy device of claim 28, said first region further comprising a first front panel and a first back band, said second region further comprising a second front panel and a second back band, wherein said first front panel is removably coupled to said second front panel and said first back band is removably coupled to said second back band.

31. The thermal compression therapy device of claim 30, wherein said first and second shoulder straps, said first and second front panels, and said first and second back bands are configured to alter the sizing of said garment to be size adjustable for a particular individual.
